(19) United States
(12) Patent Application Publication
(10) Pub. No.: US 2006/0018966 A1
(43) Pub. Date: Jan. 26, 2006

(54) Antimicrobial mesoporous silica nanoparticles

(76) Inventors: Victor Shang-Yi Lin, Ames, IA (US); Brian G. Trewyn, Ames, IA (US); Seong Huh, Ames, IA (US); Chad M. Whitman, Stanford, CA (US)

Correspondence Address: SCHWEGMAN, LUNDBERG, WOESSNER & KLUTH, 1600 TCF TOWER, 121 SOUTH EIGHT STREET, MINNEAPOLIS, MN, 55402, US

(21) Appl. No.: 10/945,545

(22) Filed: Sep. 20, 2004

Related U.S. Application Data
(63) Continuation-in-part of application No. PCT/US04/23468, filed on Jul. 21, 2004.
Application No. PCT/US04/23468 is a continuation-in-part of application No. 10/830,479, filed on Apr. 22, 2004.
(60) Provisional application No. 60/489,043, filed on Jul. 22, 2003.

(51) Int. Cl. A61K 31/44; A61K 31/4164; A61K 9/14

(52) U.S. Cl. 424/484; 514/358; 514/396

(57) ABSTRACT

Methods for preparing a series of mesoporous silicates, such as room-temperature ionic liquid (RTIL)-templated mesoporous silicate particles, with various particle morphologies are provided. Methods for preparing silicate particles with antimicrobial agents within the MSN pores is also provided. The particles can be used as controlled-release nanodevices to deliver antimicrobial agents.

### RELATED APPLICATIONS

This application is a continuation-in-part of application Ser. No. PCT/US2004/023468, filed Jul. 21, 2004, pending, which is a continuation-in-part of U.S. patent application Ser. No. 10/830,479, filed Apr. 22, 2004, pending, which claims priority under 35 U.S.C. § 119(e) to U.S. Provisional Patent Application No. 60/489,043 filed Jul. 22, 2003, which applications are incorporated herein by reference.

### GOVERNMENT FUNDING

This invention was made with Government support under NSF Contract No. CHE-0239570. The United States Government has certain rights in this invention.

### BACKGROUND OF THE INVENTION

Structurally well-defined mesoporous silica materials, such as MCM-41/48, SBA-15, MSU-n, KIT-1, and FSM-16, have recently attracted much attention for their potential applications in sensing, catalysis, and drug delivery. For MCM-41/48 materials, see Beck, J. S.; Vartuli, J. C.; Roth, W. J.; Leonowicz, M. E.; Kresge, C. T.; Schmitt, K. D.; Chu, C. T. W.; Olson, D. H.; Sheppard, E. W. J. Am. Chem. Soc. 1992, 114, 10834-10843; Kresge, C. T.; Leonowicz, M. E.; Roth, W. J.; Vartuli, J. C.; Beck, J. S. Nature (London) 1992, 359, 710-712. For SBA-15 materials, see Zhao, D.; Feng, J.; Huo, Q.; Melosh, N.; Frederickson, G. H.; Chmelka, B. F.; Stucky, G. D. Science (Wash., D. C.) 1998, 279, 548-552. For MSU-n materials, see Bagshaw, S. A.; Prouzet, E.; Pinnavaia, T. J. Science (Wash., D. C.) 1995, 269, 1242-1244. For KIT-1 materials, see Ryoo, R.; Kim, J. M.; Ko, C. H.; Shin, C. H. J. Phys. Chem. 1996, 100, 17718-17721. For MSN-16 materials, see Inagaki, S.; Koiwai, A.; Suzuki, N.; Fukushima, Y.; Kuroda, K. Bull. Chem. Soc. Jpn. 1996, 69, 1449-1457. For sensing applications, see Lin, V. S. Y.; Lai, C.-Y.; Huang, J.; Song, S.-A.; Xu, S. J. Am. Chem. Soc. 2001, 123, 11510-11511; Radu, D. R.; Lai, C.-Y.; Wiench, J. W.; Pruski, M.; Lin, V. S. Y. J. Am. Chem. Soc. 2004, 126, 1640-1641; Casasus, R.; Marcos, M. D.; Martinez-Manez, R.; Ros-Lis, J. V.; Soto, J.; Villaescusa, L. A.; Amoros, P.; Beltran, D.; Guillem, C.; Latorre, J. J. Am. Chem. Soc. 2004, 126, 8612-8613. For catalysis applications, see Huh, S.; Chen, H.-T.; Wiench, J. W.; Pruski, M.; Lin, V. S. Y. J. Am. Chem. Soc. 2004, 126, 1010-1011; Lin, V. S. Y.; Radu, D. R.; Han, M.-K.; Deng, W.; Kuroki, S.; Shanks, B. H.; Pruski, M. J. Am. Chem. Soc. 2002, 124, 9040-9041; Corma, A. Chem. Rev. 1997, 97, 2373-2419; Thomas, J. M. J. Mol. Catal. A 1999, 146, 77-85; Brunel, D.; Blanc, A. C.; Galarneau, A.; Fajula, F. Catal. Today 2002, 73, 139-152. For drug delivery applications, see Lai, C.-Y.; Trewyn, B. G.; Jeftinija, D. M.; Jeftinija, K.; Xu, S.; Jeftinija, S.; Lin, V. S. Y. J. Am. Chem. Soc. 2003, 125,4451-4459; Mal, N. K.; Fujiwara, M.; Tanaka, Y.; Taguchi, T.; Matsukata, M. Chem. Mater. 2003, 15, 3385-3394; Vallet-Regi, M.; Ramila, A.; del Real, R. P.; Perez-Pariente, J. Chem. Mater. 2001, 13, 308-311; Tourne-Peteilh, C.; Brunel, D.; Begu, S.; Chiche, B.; Fajula, F.; Lemer, D. A.; Devoisselle, J.-M. New J. Chem. 2003, 27, 1415-1418. These materials are typically synthesized by utilizing organic surfactants or block copolymers as structure-directing templates in acid- or base-catalyzed condensation of alkoxysilanes. The realization of the aforementioned applications for mesoporous silica materials greatly depends on the ability of controlling not only the intra-particle, but also the inter-particle mass-transport processes. Therefore, it is important to develop methods to regulate both the pore and particle morphology of these materials (Huh, S.; Wiench, J. W.; Yoo, J.-C.; Pruski, M.; Lin, V. S. Y. Chem. Mater. 2003, 15, 4247-4256; Huh, S.; Wiench, J. W.; Trewyn, B. G.; Song, S.; Pruski, M.; Lin, V. S. Y. Chem. Commun. (Cambridge) 2003, 2364-2365).

To develop methods to regulate both the pore and particle morphology of mesoporous silica nanoparticles (MSNs), several recent reports have focused on the utilization of other amphiphilic molecules, such as room-temperature ionic liquids (RTILs), as templates for the synthesis of mesoporous silica materials. For example, Zhou et al. have demonstrated that monolithic mesoporous silica with either wormlike pores or lamellar super-microporous structure could be prepared by using 1-alkyl-3-methylimidazolium (CₙMIM, n=the number of carbons in the alkyl chain) chloride or tetrafluoroborate, respectively, as templates (Zhou, Y.; Antonietti, M. Adv. Mater. (Weinheim, Ger.) 2003, 15, 1452-1455; Zhou, Y.; Schattka, J. H.; Antonietti, M. Nano Lett. 2004, 4, 477-481; Zhou, Y.; Antonietti, M. Chem. Mater. 2004, 16, 544-550). Also, Dai and co-workers have successfully synthesized periodic mesoporous organosilica (PMO) materials by using two different CₙMIM bromide templates in the condensation reaction of bis(triethoxysilyl)ethane (Lee, B.; Luo, H.; Yuan, C. Y.; Lin, J. S.; Dai, S. Chem. Commun. 2004, 240-241). Despite these recent advancements, no study has been reported on how the particle morphology (size and shape) could be regulated by these RTILs.

In the field of drug delivery, many site-selective deliveries, e.g., deliveries of highly toxic antitumor drugs, such as Taxol, require “zero release” before reaching the targeted cells or tissues. Unfortunately, the release of compounds from many drug delivery systems takes place immediately upon dispersion of the drug/carrier composites in water. The release mechanism of other systems, such as biodegradable polymer-based drug delivery systems, also relies on the hydrolysis-induced erosion of the carrier structure. See Uhrich, K. E., et al., Chem. Rev. 1999, 99, 3181-3198; and Langer, R. Acc. Chem. Res. 1993, 26, 537-542. Additionally, many polymeric based release systems require organic solvents for drug loading, which can trigger undesirable modifications of the structure or function of the encapsulated molecules, such as protein denaturation or aggregation. See Li, Y., Kissel, T., J. Controlled Release 1993, 27, 247-257.

The development of mesoporous silica-based carrier systems for controlled-release delivery of drugs, biocides, genes, or even proteins in vitro or in vivo is of keen interest. See Vallet-Regi, M., et al., Chem. Mater. 2001, 13, 308-311; Munoz, B., et al., Chem. Mater. 2003, 15, 500-503; Ramila, A., et al., J. Sol.-Gel Sci. Technol. 2003, 26, 1199-1202; Diaz, J. F., et al., J. Mol. Catal. B: Enzym. 1996, 2, 115-126; Han, Y.-J., et al., J. Am. Chem. Soc. 1999, 121, 9897-9898; Kisler, J. M., et al., Microporous Mesoporous Mater. 2001, 44-45, 769-774; Yiu, H. H. P., et al., Microporous Mesoporous Mater. 2001, 44-45, 763-768; and Takahashi, H., et al., Microporous Mesoporous Mater. 2001, 44-45, 755-762. Despite this current interest, there remains a need for novel carrier systems that can be used for the controlled-release delivery of antimicrobial agents in vitro or in vivo.

### SUMMARY OF THE INVENTION

The present invention provides a room temperature ionic liquid (RTIL)-templated mesoporous silicate body, as well as a micro- or a nanoparticle, having one or more pores, one or more RTIL cations within one or more of the pores of the mesoporous silicate body, and one or more functionalized organic groups in one or more of the pores. The RTIL cation can be an antimicrobial agent. The mesoporous silicate body can optionally contain any suitable and effective antimicrobial agent. The antimicrobial agent can be an antimicrobial quaternary ammonium cation, such as, for example, a RTIL cation.

The antimicrobial agent can be a biocidal quaternary ammonium salt, or “quat”, such as a (higher)alkylpyridinium cation, for example, a cetylpyridinium cation. Alternatively, the antimicrobial agent can be a 1-(higher)alkyl-3-alkylimidazolium cation, for example, a 1-tetradecyl-3-methylimidazolium cation, a 1-hexadecyl-3-methylimidazolium cation, a 1-octadecyl-3-methylimidazolium cation, or a 1-tetradecyloxymethyl-3-methylimidazolium cation. The antimicrobial agent can be a cation or a salt. Any suitable and effective counter-ion can be used with the cations described herein. A combination of antimicrobial agents can be contained in the pores of the mesoporous silicate body. The RTIL cations can diffuse from the pores of the mesoporous silicate body when in contact with a liquid that has a pH of greater than about 7, a pH of about 7.5 to about 9, or a pH of about 7.8 to about 8.5. Upon release from the pores, the antimicrobial agent can be effective against cocci, rods, or fungi. The antimicrobial agent can be effective against gram negative bacteria, gram positive bacteria, or both.

The mesoporous silicate bodies can be prepared with any suitable functionalized organic group in the one or more pores. The functionalized organic group can include an alkyl thiol, one or more amino acids, or both. The one or more amino acids can be any amino acid, including one or more selected from the group consisting of glutamic acid, histidine, and aspartic acid.

The mesoporous silicate bodies can be prepared by condensing silicates around surfactant templates. When the surfactant template is an antimicrobial ammonium species, the as-synthesized bodies can be used as delayed-release antimicrobial delivery systems because the template molecules can slowly diffuse from the pores of the bodies under physiological conditions. As such, the as-synthesized particles can be used in commercial preparations, such as a mouthwash. Other delayed-release antimicrobial delivery systems can be prepared by removing the surfactant template and re-loading the pores of the particles with antimicrobial agents, such as antimicrobial quaternary ammonium salts, zinc-containing agents, bis-biguanidine agents, or combinations thereof

A delayed-release antimicrobial delivery system can be prepared by coating the particles with a polymer. The particles can be coated by either forming covalent bonds to a polymer or by encapsulating the particles within a polymer. The polymer coating can act to slow the rate of diffusion of the RTIL cations from the pores of the mesoporous silicate body when it is in contact with a liquid. The polymer can be an adhesive, such as a bioadhesive. The adhesive can adhere the particle to the oral tissue of a mammal, such as a human, a human companion, or a farm animal, when the silicate body is contacted with the mouth of a mammal. Alternatively, adhesive can adhere the silicate body to the skin or other mucus membranes of a mammal when the when the silicate body is contacted with cells or membranes. For example, the polymer can be poly(lactic acid). Alternatively, the polymer can be, for example, an adhesive such as an alkyl vinyl ether-maleic copolymer or a poly(N-isopropylacrylamide).

The mesoporous silicate body can have an average particle diameter of about 40-100 nm, about 100-300 nm, about 300-600 nm, or about 500 nm to about 4 μm, and can have an average pore diameter of about 1 to about 4 nm, about 2 to about 3.5 nm, or about 2.5 nm. The particles can have various pre-determined shapes, including, e.g., a spheroid shape, an ellipsoid shape, a rod-like shape, or a curved cylindrical shape.

The body can contain zinc-binding amino acids. The zinc-binding amino acids can be covalently bonded to the surfaces of pores of the mesoporous silicate body. The zinc-binding amino acids can be one or more of glutamic acid, histidine, and aspartic acid, or any other amino acid that can maintain an attraction to zinc sufficient to maintain zinc within the pores of the body for an appropriate period of time. The mesoporous silicate body can contain one or more metals, metal compounds, or metal cations. The metal cation can be a zinc cation. The metal compound can be a zinc salt of an organic acid such as zinc acetate. The body can contain one or more bis-biguanidines, such as chlorhexidine, or salts thereof within one or more of the pores. The body can bind and release metal ions or metal-containing compounds.

The invention provides a pharmaceutical composition containing an effective amount of the mesoporous silicate particles described herein, in combination with a pharmaceutically acceptable diluent or carrier. The invention also provides a cosmetic composition containing the particle as described herein, in combination with a cosmetically acceptable diluent or carrier.

The invention further provides a method of treatment by inhibiting microbial growth by contacting a mammal, such as a human, companion animal, or farm animal, with an effective amount of the mesoporous silicate particles of the invention. The method includes contacting the oral tissue, the skin, or a mucus membrane of the mammal. The treatment can reduce the production of odoriferous volatile sulfur compounds in the mouth of a mammal.

The invention provides a method for synthesizing ellipsoid-, rod-, or tubular-shaped mesoporous silicate nanoparticles by co-condensing one or more tetraalkoxy-silanes and one or more room temperature ionic liquids to provide a population of mesoporous silicate particles having monodisperse particle sizes, wherein the RTIL is not a co-solvent. The mesoporous silicate particles can be prepared by co-condensing one or more tetraalkoxy-silanes and a 1-hexadecyl-3-methylimidazolium salt to provide the mesoporous silicate particles as ellipsoids, one or more tetraalkoxy-silanes and a 1-octadecyl-3-methylimidazolium salt to provide the mesoporous silicate particles as rods, or one or more tetraalkoxy-silanes and a 1-tetradecyloxymethyl-3-methylimidazolium salt to provide the mesoporous silicate particles as curved cylindrical shaped particles. One or more organo-substituted trialkoxy-silanes can also be co-condensed into the silicate body. The organo-substituted trialkoxy-silane can be, for example, a thioalkyl-substituted trialkoxy-silane.

The invention provides a method of administering an antimicrobial agent to a mammal by contacting the mammal with a RTIL-templated mesoporous silicate particle that contains a quaternary ammonium cation within one or more pores. The antimicrobial agent can be an (higher)alkylpyridinium cation or a cetylpyridinium cation. The antimicrobial agent can be a 1-(higher)alkyl-3-alkylimidazolium cation, for example, a 1-tetradecyl-3-methylimidazolium cation, a 1-hexadecyl-3-methylimidazolium cation, a 1-octadecyl-3-methylimidazolium cation, or a 1-tetradecyloxymethyl-3-methylimidazolium cation.

The mesoporous silicate particle can contain zinc-binding amino acids. The zinc-binding amino acids can be covalently bonded to the surface of pores of the mesoporous silicate particle. The zinc-binding amino acids can be, for example, one or more of glutamic acid, histidine, and aspartic acid. The mesoporous silicate particle can contain one or more metals, metal compounds, or metal cations. The metal cation can be a zinc cation. The metal compound can be a zinc salt of an organic acid such as zinc acetate. The mesoporous silicate particle can contain a bis-biguanidine or a salt thereof. The bis-biguanidine can be chlorhexidine or a salt thereof. The mesoporous silicate particle can bind and release metal ions or metal-containing compounds.

The method can include contacting the oral tissue, skin, or a mucus membrane of a mammal with the mesoporous silicate particle. The treatment can reduce the production of volatile sulfur compounds from an amount produced prior to treatment. When released from the pores, the antimicrobial agent can be effective against cocci, rods, or fungi. The antimicrobial agent can be effective against gram negative bacteria, gram positive bacteria, or both. The antimicrobial agent can be selective for a specific bacteria or fungus. A polymer can be covalently bonded to the surface of the mesoporous silicate body. The polymer can slow the rate of diffusion of the antimicrobial agent from the pores of the mesoporous silicate body when it is in contact with a liquid. The mesoporous silicate body can have a polymer covalently bonded to its surface. The polymer can be an adhesive, which can adhere the body to the oral tissue of a mammal when the when the silicate body is contacted with the mouth of a mammal. Alternatively, the adhesive can adhere the silicate body to skin cells or mucus membrane of a mammal when the when the silicate body is contacted with cells or membranes. The adhesive can be an alkyl vinyl ether-maleic copolymer, poly(N-isopropylacrylamide), or any other suitable and effective adhesive.

The invention provides an antimicrobial delivery system that allows for delayed release of antibacterial agents from a single application of mesoporous silicate particles. The system can contain one or more mesoporous silicate particles having one or more pores, one or more antimicrobial agents within one or more pores, wherein the mesoporous silicate particles release the antimicrobial agents from the pores or the surface of the mesoporous silicate particles over an extended period of time. The antimicrobial delivery system can also contain one or more amino acids covalently bonded to the pores or the surface of the mesoporous silicate particles, wherein the amino acid influences the release rate of an antimicrobial agent. The antimicrobial agent can be selective for a specific bacteria or fungus. The antimicrobial agent can be selective for gram negative bacteria, gram positive bacteria, or both. The particle can have a polymer covalently bonded to the surface of the mesoporous silicate particles. The polymer can be a coating or an adhesive. The polymer can be an alkyl vinyl ether-maleic copolymer, poly(N-isopropylacrylamide), poly(lactic acid), or any other suitable and effective polymer.

The invention provides a method of reducing oral volatile sulfur compounds by contacting a mammal with an antimicrobial controlled-release composition that contains a silicate body as described herein. The method of reducing oral volatile sulfur compounds can be used in conjunction with an oral rinse, such as a mouthwash.

Mesoporous silicate particles of the invention can be used in medical therapy. Medical therapies for which the mesoporous silicate particles may be used include any therapy employs an antimicrobial agent, particularly a microbial agent that is delivered to the mouth, skin, or a mucus membrane. Such medical therapies include, e.g., treating inflammation, infection, cell senescence, skin disorders, radiation dermatitis, sunburn, oral malodor, and related conditions. The mesoporous silicate particles can also be used to prepare a medicament for treatment of, e.g., inflammation, infection, cell senescence, skin disorders, radiation dermatitis, sunburn, oral malodor, and related conditions. Such medicaments can also include a physiologically acceptable diluent or carrier.

### BRIEF DESCRIPTION OF THE DRAWINGS

FIG. 1. illustrates chemical structures of 1-tetradecyl-3-methylimidazolium bromide (C₁₄MIMBr), 1-hexadecyl-3-methylimidazolium bromide (C₁₆MIMBr), 1-octadecyl-3-methylimidazolium bromide (C₁₈MIMBr), 1-tetradecyloxymethyl-3-methylimidazolium chloride (C₁₄CMIMCl), and cetylpyridinium bromide (CPBr).

FIG. 2. illustrates a schematic representation of the controlled release process of CₙMIM-MSN and its antibacterial activity against E. coli.

FIG. 3. illustrates transmission electron micrographs of CₙMIM-MSN materials: (a) C₁₄MIM-MSN, (b) C₁₆MIM-MSN, (c) C₁₈MIM-MSN, and (d) C₁₄OCMIM-MSN.

FIG. 4. illustrates low angle powder X-ray diffraction patterns of RTIL-removed CₙMIM-MSN materials. (a) C₁₄MIM-MSN, (b) C₁₆MIM-MSN, (c) C₁₈MIM-MSN, (d) C₁₄OCMIM-MSN, (e) CP-MSN.

FIG. 5. illustrates a disk diffusion assay of 15 mM C₁₆MIM-MSN (a), C₁₄OCMIM-MSN (b), phosphate buffer (c), and CP-MSN (d) on a lawn of E. coli K12. The red arrow points to an area of microbial lawn and the blue arrow points to the zone of clearing caused by the diffusion of RTIL.

FIG. 6. illustrates a histogram of the antibacterial activity of CₙMIM-MSNs against E. coli K12 at 25° C. (a) and 37° C. (b). Four samples were measured at each temperature: CP-MSN (vertical dashes), C₁₆MIM-MSN (crossed lines), C₁₄OCMIM-MSN (slanted lines), RTIL-removed C₁₆MIM-MSN (horizontal lines), and blank control (no silica material) (vertical lines).

### DETAILED DESCRIPTION OF THE INVENTION

As used herein, the term “mesoporous silicate” refers to a mesoporous structure formed by the acid or base catalyzed condensation of a silicon containing material around a surfactant template, forming typically uniform channel structures. As used herein, the terms “mesoporous silicate”, “mesoporous silicate body”, “mesoporous silicate particle”, and “mesoporous silicate nanoparticle” (MSN) can be used interchangeably. The mesoporous silicate body can have an average particle diameter of about 40-100 nm, about 100-300 nm, about 300-600 nm, or about 500 nm to about 4 μm, and can have an average pore diameter of about 1 to about 4 nm, about 2 to about 3.5 nm, or about 2.5 nm. The particles can have various pre-determined shapes, including, e.g., a spheroid shape, an ellipsoid shape, a rod-like shape, or a curved cylindrical shape.

As used herein, the term “room temperature ionic liquid” (RTIL) refers to a binary ionic salt that is a liquid at temperatures of about −100° C. to about 100° C., wherein the cation is an organic cation. The organic cations of room temperature ionic liquids as described herein include alkylammonium and alkylphosphonium cations, and heterocyclic cations, such as N-alkylpyridinium, and N,N′-dialkylimidazolium. As used herein, an organic cation is a carbon-containing species that contains a positively charged heteroatom. A RTIL cation is an organic cation that can be combined with an appropriate anion to form a room temperature ionic liquid. Some common RTIL anions include tetrafluoroborate, hexafluorophosphate, tetrachloroaluminate, trifluoroacetate, and halides, such as fluoride, chloride, bromide, and iodide. One procedure for preparing a RTIL is to reflux an alkyl halide with a heterocycle that contains a sufficiently nucleophilic atom, such as nitrogen or phosphorus, to produce an ionic liquid composed of an alkylated organic cation and a halogen anion (see also Welton, T., Chem. Rev. 1999, 99(8), 2071-2084; and Dupont, J. et al. Chem. Rev. 2002, 102(10), 3667-3692).

As used herein, the term “antimicrobial agent” refers to any agent that kills, inhibits the growth of, or prevents the growth of a bacteria, fungus, yeast, or virus. Antimicrobial agents include pharmaceutical agents, biocidal or pesticidal agents (e.g. insecticides, herbicides, and rodentacides), antibacterial agents, antifingal agents, and antiviral agents (see U.S. Pat. No. 4,950,758). Quaternary ammonium compounds (“quats”) can be antimicrobial agents. A quat is a positively charged nitrogen atoms that is bonded to four organic groups. A quat can have any suitable counter-ion when it forms a salt. Quats typically have at least one higher(alkyl) substituent. As used herein, higher(alkyl) refers to a C₁₀-C₂₂(alkyl) group, optionally interrupted on the carbon chain with 1-3 ether linkages.

Antimicrobial agents that can be incorporated into the pores of the mesoporous silicate body include, but are not limited to, antibiotics such as vancomycin, bleomycin, pentostatin, mitoxantrone, mitomycin, dactinomycin, plicamycin and amikacin. Other antimicrobial agents include antibacterial agents such as 2-p-sulfanilyanilinoethanol, 4,4′-sulfinyldianiline, 4-sulfanilamidosalicylic acid, acediasulfone, acetosulfone, amikacin, amoxicillin, amphotericin B, ampicillin, apalcillin, apicycline, apramycin, arbekacin, aspoxicillin, azidamfenicol, azithromycin, aztreonam, bacitracin, bambermycin(s), biapenem, brodimoprim, butirosin, capreomycin, carbenicillin, carbomycin, carumonam, cefadroxil, cefamandole, cefatrizine, cefbuperazone, cefclidin, cefdinir, cefditoren, cefepime, cefetamet, cefixime, cefmenoxime, cefininox, cefodizime, cefonicid, cefoperazone, ceforanide, cefotaxime, cefotetan, cefotiam, cefozopran, cefpimizole, cefpiramide, cefpirome, cefprozil, cefroxadine, ceftazidime, cefteram, ceftibuten, ceftriaxone, cefuzonam, cephalexin, cephaloglycin, cephalosporin C, cephradine, chloramphenicol, chlortetracycline, ciprofloxacin, clarithromycin, clinafloxacin, clindamycin, clindamycin phosphate, clomocycline, colistin, cyclacillin, dapsone, demeclocycline, diathymosulfone, dibekacin, dihydrostreptomycin, dirithromycin, doxycycline, enoxacin, enviomycin, epicillin, erythromycin, flomoxef, fortimicin(s), gentamicin(s), glucosulfone solasulfone, gramicidin S, gramicidin(s), grepafloxacin, guamecycline, hetacillin, imipenem, isepamicin, josamycin, kanamycin(s), leucomycin(s), lincomycin, lomefloxacin, lucensomycin, lymecycline, meclocycline, meropenem, methacycline, micronomicin, midecamycin(s), minocycline, moxalactam, mupirocin, nadifloxacin, natamycin, neomycin, netilmicin, norfloxacin, oleandomycin, oxytetracycline, p-sulfanilylbenzylamine, panipenem, paromomycin, pazufloxacin, penicillin N, pipacycline, pipemidic acid, polymyxin, primycin, quinacillin, ribostamycin, rifamide, rifampin, rifamycin SV, rifapentine, rifaximin, ristocetin, ritipenem, rokitamycin, rolitetracycline, rosaramycin, roxithromycin, salazosulfadimidine, sancycline, sisomicin, sparfloxacin, spectinomycin, spiramycin, streptomycin, succisulfone, sulfachrysoidine, sulfaloxic acid, sulfamidochrysoidine, sulfanilic acid, sulfoxone, teicoplanin, temafloxacin, temocillin, tetracycline, tetroxoprim, thiamphenicol, thiazolsulfone, thiostrepton, ticarcillin, tigemonam, tobramycin, tosufloxacin, trimethoprim, trospectomycin, trovafloxacin, tuberactinomycin and vancomycin. Antimicrobial agents can also include anti-fungals, such as amphotericin B, azaserine, candicidin(s), chlorphenesin, dermostatin(s), filipin, fungichromin, mepartricin, nystatin, oligomycin(s), perimycin A, tubercidin, imidazoles, triazoles, and griesofulvin. Any suitable and effective antimicrobial agent that can be loaded into the pores of the mesoporous silicate body can be employed.

The present invention provides a room temperature ionic liquid (RTIL)-templated mesoporous silicate body, as well as a micro- or a nanoparticle, having one or more pores, one or more RTIL cations within one or more of the pores of the mesoporous silicate body, and one or more functionalized organic groups in one or more of the pores. The RTIL cation can be an antimicrobial agent. The mesoporous silicate body can optionally contain any suitable and effective antimicrobial agent. The antimicrobial agent can be an antimicrobial quaternary ammonium cation, such as, for example, a RTIL cation.

The antimicrobial agent can be a biocidal quaternary ammonium salt, or “quat”, such as a (higher)alkylpyridinium cation, for example, a cetylpyridinium cation. Alternatively, the antimicrobial agent can be a 1-(higher)alkyl-3-alkylimidazolium cation, for example, a 1-tetradecyl-3-methylimidazolium cation, a 1-hexadecyl-3-methylimidazolium cation, a 1-octadecyl-3-methylimidazolium cation, or a 1-tetradecyloxymethyl-3-methylimidazolium cation. The antimicrobial agent can be a cation or a salt. Any suitable and effective counter-ion can be used with the cations described herein. A combination of antimicrobial agents can be contained in the pores of the mesoporous silicate body. The RTIL cations can diffuse from the pores of the mesoporous silicate body when in contact with a liquid that has a pH of greater than about 7, a pH of about 7.5 to about 9, or a pH of about 7.8 to about 8.5. Upon release from the pores, the antimicrobial agent can be effective against cocci, rods, or fungi. The antimicrobial agent can be effective against gram negative bacteria, gram positive bacteria, or both.

The mesoporous silicate bodies can be prepared with any suitable functionalized organic group in the one or more pores. The functionalized organic group can include an alkyl thiol, one or more amino acids, or both. The one or more amino acids can be any amino acid, including one or more selected from the group consisting of glutamic acid, histidine, and aspartic acid.

The mesoporous silicate bodies can be prepared by condensing silicates around surfactant templates. When the surfactant template is an antimicrobial ammonium species, the as-synthesized bodies can be used as delayed-release antimicrobial delivery systems because the template molecules can slowly diffuse from the pores of the bodies under physiological conditions. As such, the as-synthesized particles can be used in commercial preparations, such as a mouthwash. Other delayed-release antimicrobial delivery systems can be prepared by removing the surfactant template and re-loading the pores of the particles with antimicrobial agents, such as antimicrobial quaternary ammonium salts, zinc-containing agents, bis-biguanidine agents, or combinations thereof.

A delayed-release antimicrobial delivery system can be prepared by coating the particles with a polymer. The particles can be coated, either by forming covalent bonds to a polymer or by encapsulating the particles within a polymer. The polymer coating can act to slow the rate of diffusion of the RTIL cations from the pores of the mesoporous silicate body when it is in contact with a liquid. The polymer can be an adhesive, such as a bioadhesive. The adhesive can adhere the particle to the oral tissue of a mammal, such as a human, a human companion, or a farm animal, when the silicate body is contacted with the mouth of a mammal. Alternatively, adhesive can adhere the silicate body to the skin or other mucus membranes of a mammal when the when the silicate body is contacted with cells or membranes.

The polymer can be any suitable and effective polymer that, when covalently bound to the surface of the silicate body, acts to slow the diffusion of RTIL cations from the pores. One example of a polymer coating is poly(lactic acid). Additionally, an adhesive can be suitably prepared using a silicone based pressure sensitive adhesive, such as a (polydimethyl-siloxane-silicate resin) copolymer adhesive depicted by the following formula: 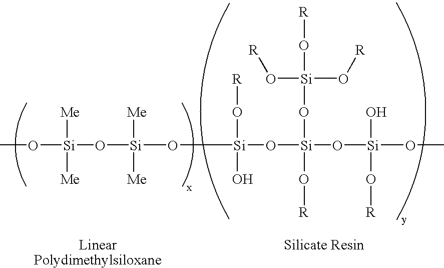
wherein R is —Si(CH₃)₃, and x and y represent independent numbers of repeating units sufficient to provide the desired properties in the adhesive polymer or other polymer layers.

For example, monomers of adhesive polymer products or amine-resistant adhesive polymer products sold by Dow Coming, such as the ones sold under the designations of DC-355, Bio-PSA and X7-2920 medical adhesives, are suitable for use in making the adhesive layer. The adhesive polymer must be biologically acceptable and chemically compatible with other components when used in a delivery system. Certain polyacrylic adhesive polymers in the form of an alkyl ester, amide, free acid, or the like or polyisobutylene adhesive polymers can also be used to covalently bond to, or to coat, the mesoporous silicate particles. Illustrative of suitable adhesive polymers for use in making the adhesive polymer layer are shown by the following formulas: 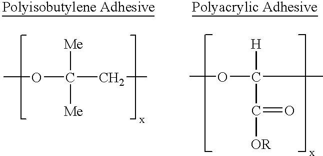
wherein x represents the number of repeating units sufficient to provide the desired properties in the adhesive polymer and R is H or (C₁-C₈)lower alkyl, including ethyl, propyl, butyl, hexyl, and branched isomers such as 2-ethylhexyl. One type of adhesive layer that can be used in conjunction with the mesoporous silicate bodies is a pressure sensitive adhesive. Other suitable hypoallergenic pressure-sensitive contact adhesive compositions can also be used. Some specific adhesives include, e.g., an alkyl vinyl ether-maleic copolymer, poly(N-isopropylacrylamide) (NiPAAM), or any other suitable and effective adhesive.

The particles can bind and release antimicrobial agents, metals, metal ions, or metal-containing compounds. The antimicrobial agent can be a quaternary ammonium compound. The particles can optionally contain zinc-binding amino acids such as, for example, one or more of glutamic acid, histidine, and aspartic acid, or any other amino acid that can maintain an attraction to zinc sufficient to maintain zinc within the pores of the particle for an appropriate period of time. The zinc-binding amino acids can be covalently bonded to the surface of pores of the mesoporous silicate body through an organic moiety. The mesoporous silicate body can contain one or more metals, metal compounds, or metal cations. The metal cation can be, for example, a zinc cation. The metal compound can be a zinc salt of an organic acid such as zinc acetate. The particle can also contain one or more bis-biguanidines within one or more pores. The bis-biguanidine can be, for example, chlorhexidine, or a salt thereof.

As used herein, the term “amino acid,” comprises the residues of the natural amino acids (e.g. Ala, Arg, Asn, Asp, Cys, Glu, Gln, Gly, His, Hyl, Hyp, Ile, Leu, Lys, Met, Phe, Pro, Ser, Thr, Trp, Tyr, and Val) in D or L form, as well as unnatural amino acids (e.g. phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine, ornithine, citruline, α-methyl-alanine, para-benzoylphenylalanine, phenylglycine, propargylglycine, sarcosine, and tert-butylglycine). The term also comprises natural and unnatural amino acids bearing an amino protecting group (e.g. acetyl or benzyloxycarbonyl), as well as natural and unnatural amino acids protected at the carboxy terminus (e.g. as a (C₁-C₆)alkyl, phenyl or benzyl ester or amide). Other suitable amino and carboxy protecting groups are known to those skilled in the art (See for example, T. W. Greene, Protecting Groups In Organic Synthesis; Wiley: N.Y., 1981, and references cited therein).

The invention provides a pharmaceutical composition containing an effective amount of the mesoporous silicate particles described herein, in combination with a pharmaceutically acceptable diluent or carrier. The invention also provides a cosmetic composition containing the particle as described herein, in combination with a cosmetically acceptable diluent or carrier.

The invention further provides a method of treatment by inhibiting microbial growth by contacting a mammal, such as a human, companion animal, or farm animal, with an effective amount of the mesoporous silicate particles of the invention. The method includes contacting the oral tissue, the skin, or a mucus membrane of the mammal. The treatment can reduce the production of odoriferous volatile sulfur compounds in the mouth of a mammal.

The invention provides a method for synthesizing ellipsoid-, rod-, or tubular-shaped mesoporous silicate nanoparticles by co-condensing one or more tetraalkoxy-silanes and one or more room temperature ionic liquids to provide a population of mesoporous silicate particles having monodisperse particle sizes, wherein the RTIL is not a co-solvent. The mesoporous silicate particles can be prepared by co-condensing one or more tetraalkoxy-silanes and a 1-hexadecyl-3-methylimidazolium salt to provide the mesoporous silicate particles as ellipsoids, one or more tetraalkoxy-silanes and a 1-octadecyl-3-methylimidazolium salt to provide the mesoporous silicate particles as rods, or one or more tetraalkoxy-silanes and a 1-tetradecyloxymethyl-3-methylimidazolium salt to provide the mesoporous silicate particles as curved cylindrical shaped particles. One or more organo-substituted trialkoxy-silanes can also be co-condensed into the particle. The organo-substituted trialkoxy-silane can be, for example, a thioalkyl-substituted trialkoxy-silane.

The invention provides a method of administering an antimicrobial agent to a mammal by contacting the mammal with a RTIL-templated mesoporous silicate particle that contains a quaternary ammonium cation within one or more pores. The antimicrobial agent can be an (higher)alkylpyridinium cation or a cetylpyridinium cation. The antimicrobial agent can be a 1-(higher)alkyl-3-alkylimidazolium cation, for example, a 1-tetradecyl-3-methylimidazolium cation, a 1-hexadecyl-3-methylimidazolium cation, a 1-octadecyl-3-methylimidazolium cation, or a 1-tetradecyloxymethyl-3-methylimidazolium cation.

The mesoporous silicate particle can contain zinc-binding amino acids. The zinc-binding amino acids can be covalently bonded to the surface of pores of the mesoporous silicate particle. The zinc-binding amino acids can be, for example, one or more of glutamic acid, histidine, and aspartic acid. The mesoporous silicate particle can contain one or more metals, metal compounds, or metal cations. The metal cation can be a zinc cation. The metal compound can be a zinc salt of an organic acid such as zinc acetate. The mesoporous silicate particle can contain a bis-biguanidine or a salt thereof. The bis-biguanidine can be chlorhexidine or a salt thereof. The mesoporous silicate particle can bind and release metal ions or metal-containing compounds.

The method can include contacting the oral tissue, skin, or a mucus membrane of a mammal with the mesoporous silicate particle. The treatment can reduce the production of volatile sulfur compounds from an amount produced prior to treatment. When released from the pores, the antimicrobial agent can be effective against cocci, rods, or fungi. The antimicrobial agent can be effective against gram negative bacteria, gram positive bacteria, or both. The antimicrobial agent can be selective for a specific bacteria or fungus. A polymer can be covalently bonded to the surface of the mesoporous silicate body. The polymer can slow the rate of diffusion of the antimicrobial agent from the pores of the mesoporous silicate body when the particle is in contact with a liquid. The mesoporous silicate body can have a polymer covalently bonded to its surface. The polymer can be an adhesive, which can adhere the body to the oral tissue of a mammal when the when the silicate body is contacted with the mouth of a mammal. Alternatively, the adhesive can adhere the particle to skin cells or mucus membrane of a mammal when the when the silicate body is contacted with cells or membranes. The adhesive can be an alkyl vinyl ether-maleic copolymer, poly(N-isopropylacrylamide), or any other suitable and effective adhesive.

Antimicrobial Delivery System

The invention provides an antimicrobial delivery system that allows for delayed release of antibacterial agents from a single application of mesoporous silicate particles. The system can contain one or more mesoporous silicate particles having one or more pores, one or more antimicrobial agents within one or more pores, wherein the mesoporous silicate particles release one or more of the antimicrobial agents from the pores or the surface of the mesoporous silicate particles over an extended period of time. An extended period of time can be up to about 4 hours, up to about 8 hours, up to about 24 hours, up to about 2 days, or up to about 7 days. The type of mesoporous silicate body used in the delivery system, the type of optional organic components in the pores of the body, and the nature and thickness of an optional polymer coating of the body determines the amount time over which the antimicrobial agents are released from the delivery devise.

The antimicrobial delivery system can also contain one or more amino acids covalently bonded to the pores or the surface of the mesoporous silicate particles, wherein the amino acid influences the release rate of an antimicrobial agent. The antimicrobial agent can be selective for a specific bacteria or fungus. The antimicrobial agent can be selective for gram negative bacteria, gram positive bacteria, or both. The particle can have a polymer covalently bonded to the surface of the mesoporous silicate particles. The polymer can be a coating or an adhesive. The polymer can be an alkyl vinyl ether-maleic copolymer, poly(N-isopropylacrylamide) or poly(lactic acid).

The invention provides a method of reducing oral volatile sulfur compounds by contacting a mammal with an antimicrobial controlled-release composition that contains a mesoporous silicate body as described herein. The method of reducing oral volatile sulfur compounds can be used in conjunction with an oral rinse, such as a mouthwash.

Mesoporous silicate particles of the invention can be used in medical therapy. Medical therapies for which the mesoporous silicate particles may be used include any therapy employs an antimicrobial agent, particularly a microbial agent that is delivered to the mouth, skin, or a mucus membrane. Such medical therapies include, e.g., treating inflammation, infection, cell senescence, skin disorders, radiation dermatitis, sunburn, oral malodor, and related conditions. The mesoporous silicate particles can also be used to prepare a medicament for treatment of, e.g., inflammation, infection, cell senescence, skin disorders, radiation dermatitis, sunburn, oral malodor, and related conditions. Such medicaments can also include a physiologically acceptable diluent or carrier.

Mesoporous Silicates

Mesoporous silicate particles can be prepared by various methods such as by co-condensing one or more tetraalkoxy-silanes and one or more organo-substituted trialkoxy-silanes to provide a population of mesoporous silicate particles having monodisperse particle sizes and preselected particle shapes, wherein the substituted trialkoxy-silane is not a co-solvent. The mesoporous silicate particles can be prepared by co-condensing one or more tetraalkoxy-silanes and one or more (3-cyanopropyl) trialkoxy-silanes to provide the mesoporous silicate particles as nanorods. Any suitable and effective tetraalkoxy-silane and alkyl-trialkoxy-siliane can be employed. Many such silanes are described in, e.g., Aldrich Handbook of Fine Chemicals, 2003-2004 (Milwaukee, Wis.).

The mesoporous silicates can be formed around surfactant micelles of ammonium salts in water. The ammonium salts can be room temperature ionic liquids or C₁₀-C₂₀ alkyl(trialkyl)ammonium salts. The mesoporous silicates can be prepared from surfactant micelles in water, followed by introduction into the solution of an alkyl orthosilicate, such as tetraethylorthosilicate (TEOS), and optionally one or more functionalized silanes, such as one or more mercaptoalkyl-, chloroalkyl-, isocyanate-, aminoalkyl-, carboxyalkyl-, sulfonylalkyl-, arylalkyl-, alkynyl-, or alkenyl-silanes, wherein the (C₂-C₁₀)alkyl chain is optionally interrupted by —S—S—, amido (—C(═O)NR—), —O—, ester (—C(═O)O—), and the like. For example, functionalized silanes can be, e.g., 3-mercaptopropyl-trimethoxysilane (MPTMS) or 3-isocyanatoprypyl-triethoxysilane (ICPTES). The aqueous mixture is stirred at moderate temperatures until the silicate precipitates, after which it is collected and dried.

The surfactant “template” can be removed from the pores of the ordered silicate matrix, for example, by refluxing the silicate in aqueous-alcoholic HCl. The remaining solvent can be removed from the pores of the silicate by placing it under high vacuum. Functional groups incorporated on the surface of the pores can be quantified and used as linker moieties to bind metals, metal cations, metal compounds, and antimicrobial agents. Functional groups incorporated on the surface of the pores can also be further modified for improved binding to metals, metal cations, metal compounds, and antimicrobial agents. Typical modifications include covalently bonding amino acids to the functional groups linked to the surfaces of the pores. The polarity of the interior of the pores can also be adjusted by adding other functionalized silanes to the reaction mixture, including ones comprising non-polar inert groups such as aryl, perfluoroalkyl, alkyl, arylalkyl and the like. The exterior of the silicate matrix can be functionalized by grafting organic moieties comprising functional groups thereto. These groups can in turn be employed to link the particles to polymers that can prolong the release time of agents within the pores, or that can adhere the particles to cells of the body of a mammal.

Loading Antimicrobial Agents

Antimicrobial agents can typically be loaded into MSNs by contact with a solution of the agent to be taken up by the particle. Agents can typically be loaded by allowing the agent to react with, or be attracted to, groups on the interior surface of the pores under conditions suitable to allow the agent to associate. In one embodiment, the mesoporous silicates can be stirred in ethanol for a period of time sufficient to load the material into the pores. Any suitable and effective solvent can be employed in this particular manner of pore loading.

Delivery of Loaded Particles to Target Site

The loaded particles of the invention can be delivered to the target site of a mammal by any suitable means, which can be selected based on the nature of the target site and the antimicrobial agent. For uses in vivo the particles can be administered orally, topically or by injection using conventional means.

Pharmaceutical and Cosmetic Compositions

The mesoporous silica particles of the invention that comprise therapeutic or cosmetic agents can be formulated as pharmaceutical or cosmetic compositions and administered to a mammalian host, such as a human patient in a variety of forms adapted to the chosen route of administration, i.e., orally or parenterally, by intravenous, intramuscular, topical or subcutaneous routes.

Thus, the mesoporous silica particles may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible gum, tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The gum, tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, gums, tablets, pills, or capsules may be coated with gelatin, wax, shellac or sugar and the like. A syrup or elixir may contain the active article, sucrose or fructose as a sweetening agent, methyl and propylparabens as preservatives, a dye and flavoring such as cherry or orange flavor. Of course, any material used in preparing any unit dosage form should be pharmaceutically or cosmetically acceptable and substantially non-toxic in the amounts employed. In addition, the active article may be incorporated into sustained-release preparations and devices.

The mesoporous silica particles may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the mesoporous silica particles of the invention in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the mesoporous silica particles will generally be administered as compositions or formulations, in combination with a dermatologically acceptable carrier, which may be a solid or a liquid, or a combination thereof.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the mesoporous silica particles of the invention to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

The invention will now be illustrated by the following non-limiting Examples.

### EXAMPLES

General Reagents and Materials. Reagents were purchased from commercial suppliers and used as received, unless otherwise stated. Nanopure water (18.1 MHz) prepared from a Bamstead E-pure water purification system was employed throughout.

### Example 1

### Synthesis of MCM41-Type RTIL-Templated Mesoporous Silica Nanosphere with Organo-Functionality

Mesoporous silica particles with organo-functionalized groups covalently bonded to the pores can be prepared by the procedure described below. Any suitable organic group can be incorporated by varying the organic group attached to a trialkoxy-silane. The following example describes the use of mercaptopropyl-trimethoxysilane (MPTMS) to obtain a mercaptopropyl-derivatized mesoporous silica nanosphere material (thiol-MSN). Suitable variations of the procedure can be used, such as those described by Lin, V. S.-Y., et al., J. Am. Chem. Soc. 2001, 123, 11510-11511; and Lin, V. S.-Y., et al., J. Am. Chem. Soc. 2002, 124, 9040-9041. RTILs can be used in place of the ammonium salt to prepare RTIL-templated MSNs.

N-Cetyltrimethylammonium bromide (CTAB, 1.00 g, 2.74×10⁻³ mol) was dissolved in 480 mL of Nanopure water. NaOH(aq) (2.00 M, 3.50 mL) was added to CTAB solution, followed by adjusting the solution temperature to 353 K. TEOS (5.00 mL, 2.57×10⁻² mol) was introduced dropwise to the surfactant solution, followed by the dropwise addition of MPTMS (0.97 mL, 5.13×10⁻³ mol). The mixture was allowed to stir for 2 hours to give white precipitates (as synthesized thiol-Sphere). The solid product was filtered, washed with deionized water and methanol, and dried in air.

To remove the surfactant template (CTAB), 1.50 g of as-synthesized thiol-Sphere was refluxed for 24 hours in a solution of 9.00 mL of HCl (37.4%) and 160.00 mL of methanol followed by extensive washes with deionized water and methanol. The resulting surfactant-removed thiol-MSN material was placed under high vacuum to remove the remaining solvent in the mesopores.

The chemically accessible thiol group surface coverage of the thiol-MSN material was quantified to be 7.64×10⁻⁴ mol/g using the method described by Lin, V. S.-Y., et al., J. Am. Chem. Soc. 2001, 123, 11510-11511. The purified thiol-MSN material (1.00 g) was treated with a methanol solution (60.00 mL) of 2-(pyridyldisulfanyl)-ethylamine (PDEA) (9.12×10⁻⁴ mol, prepared as described by Ebright, Y. W., et al., Bioconjugate Chem. 1996, 7, 380-384) at room temperature for 24 hours under vigorous stirring to undergo the desired disulfide bond exchange reaction. The resulting MSN material with 2-(propyldisulfanyl)ethylanine functionality was filtered and washed with methanol and dried in air.

### Example 2

### Morphology Control

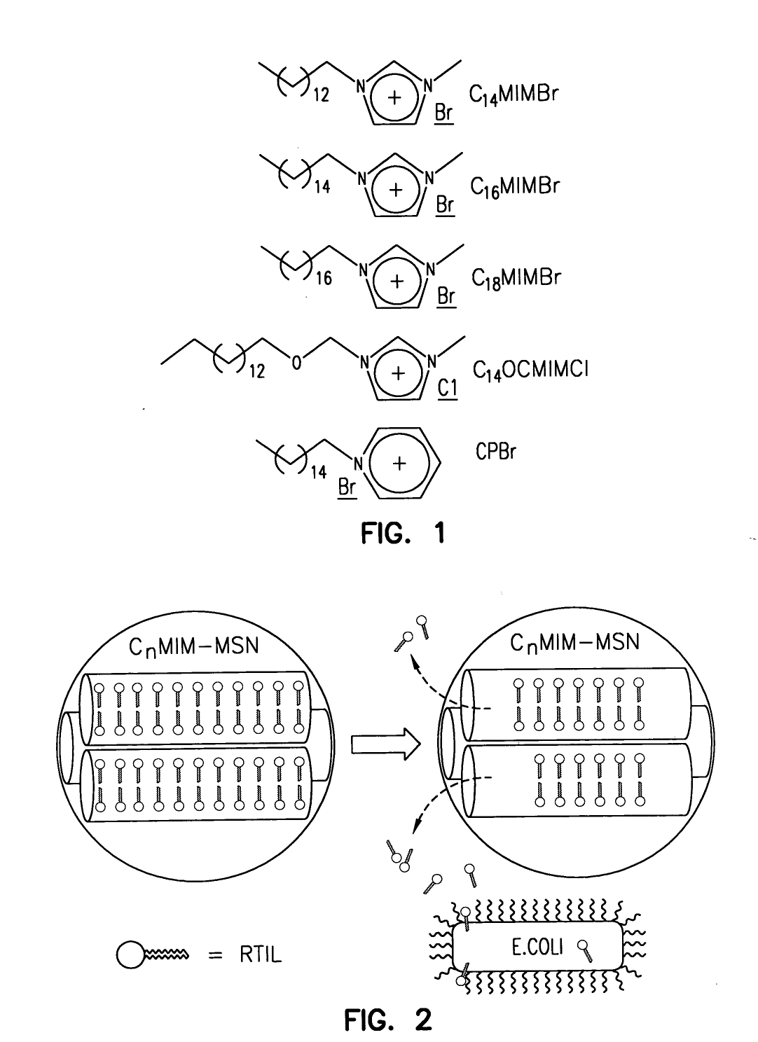

The synthesis and characterization of a series of mesoporous silica nanoparticle (MSN) materials with various porous structures and particle shapes is described herein. Particle shapes such as spheres, ellipsoids, rods, and tubes can be prepared by using different RTIL templates, such as 1-tetradecyl-3-methylimidazolium bromide (C₁₄MIMBr), 1-hexadecyl-3-methylimidazolium bromide (C₁₆MIMBr), 1-octadecyl-3-methylimidazolium bromide (C₁₈MIMBr), 1-tetradecyloxymethyl-3-methylimidazolium chloride (C₁₄OCMIMCl), and cetylpyridinium bromide (CPBr), respectively (see FIG. 1).

The C₁₄MIMBr, C₁₆MIMBr, and C₁₈MIMBr RTILs were prepared by reacting 1-methylimidazole (50 mmol) with 50 mmol of 1-bromo-tetradecane, 1-bromo-hexadecane, and 1-bromo-octadecane, respectively, at 90° C. for 48 hours. The products were purified by recrystallization in THF. The resulting white crystals were collected by filtration, and dried under vacuum at room temperature. The C₁₄OCMIMCl was prepared via a literature procedure (Pernak, J.; Sobaszkiewicz, K.; Mirska, I. Green Chem. 2003, 5, 52-56). The CPBr was commercially available. In a typical procedure for the syntheses of the CₙMIM-MSN materials, a selected CₙMIM RTIL (2.74 mmol) was first dissolved in 480 mL of 15 mM NaOH(aq). The solution was heated to 80° C., followed by a dropwise addition of tetraethyl orthosilicate (22.4 mmol) and stirred for 2 hours to yield the desired CₙMIM-MSN material.

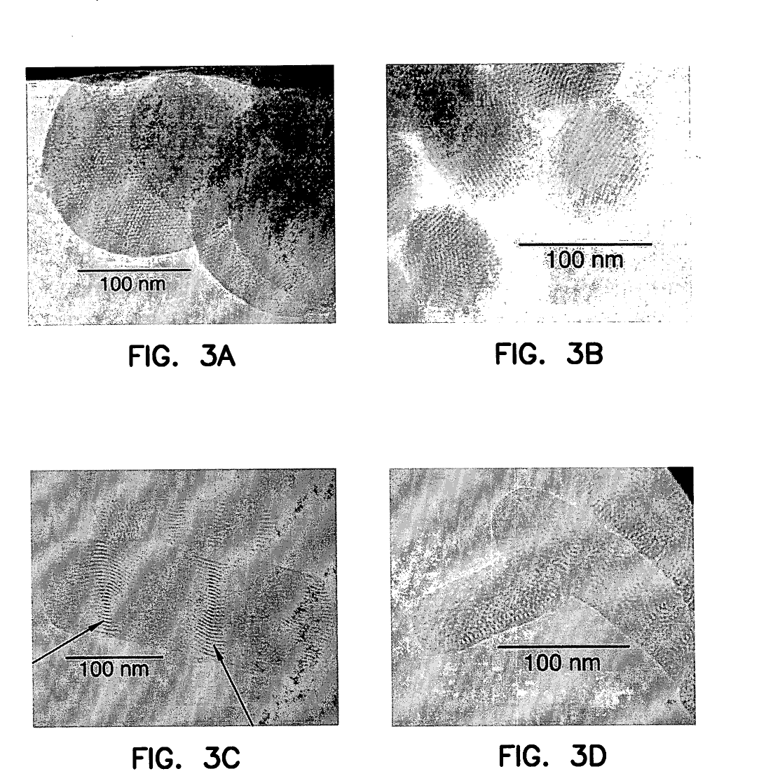

To characterize the mesoporous structures of the CₙMIM-MSNs, the CₙMIM ionic liquid molecules were extracted from the mesopores by refluxing the as-synthesized CₙMIM-MSN (500 mg) in 200 mL of methanolic solution of HCl (520 mM) for 48 hours. As revealed by the transmission electron micrographs (TEM) in FIG. 3, the CₙMIM-MSNs synthesized with the four different RTIL templates exhibited different particle morphologies. For example, the C₁₄MIM-MSN material showed spherical particles with diameters ranging from 100 to 300 nm, as depicted in FIG. 3a. Interestingly, upon replacing the C₁₄MIMBr with other structurally similar RTILs, such as C₁₆MIMBr, and C₁₈MIMBr (FIGS. 3b, c), the shapes of the MSN materials transformed into ellipsoids and rods, respectively. Furthermore, substituting the C₁₆MIM template with a similar sized C₁₄OCMIM RTIL gave rise to a MSN material (FIG. 3d) consisted of tubular shaped particles.

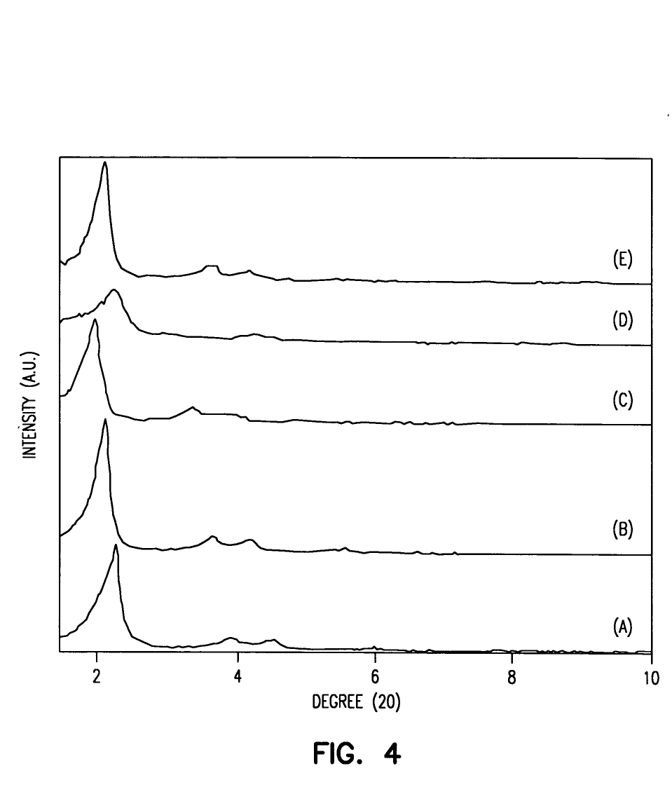

The pore morphologies of the CₙMIM-templated MSNs were determined by nitrogen adsorption-desorption surface analysis (BET isotherms and BJH pore size distributions), TEM (FIG. 3), and powder X-ray diffraction (XRD) spectroscopy. All four CₙMIM-MSN materials exhibited type IV BET isotherms. As the organic region of the RTIL increases in length the BJH average pore diameter of these materials also increases as summarized in Table 1. Hexagonally packed mesoporous channels were clearly observed in the TEM micrographs of the C₁₄MIM- and C₁₆MIM-MSNs (FIGS. 3a, b). Also, both materials exhibited diffraction patterns characteristic of hexagonal MCM-41 silicas, including (100), (110), (200), and (210) peaks as depicted in FIGS. 4a and 4b.

TABLE 1
Nitrogen sorption data of RTIL-MSN material.

| | BET surface area | Pore volume | Average Pore |
|---|---|---|---|
| | (m²/g) | (cm³/g) | Diameter (Å) |
| C₁₄MIM-MSN | 729 | 0.664 | 27.1 |
| C₁₆MIM-MSN | 924 | 0.950 | 30.3 |
| C₁₈MIM-MSN | 893 | 0.995 | 32.7 |
| C₁₄OCMIM-MSN | 639 | 0.695 | 26.1 |
| CP-MSN | 1091 | 1.41 | 27.2 |

A pseudo-moire rotational pattern of mesopores was observed in the TEM micrograph of the C₁₈MIM-MSN material, where parallel mesopores are twisted in a helical nature along the long axis of the nanorods. This pore morphology is structurally similar to a chiral mesoporous silica material recently reported by Tatsumi and co-workers (Che, S.; Liu, Z.; Ohsuna, T.; Sakamoto, K.; Terasaki, O.; Tatsumi, T. Nature (London) 2004, 429, 281-284). In contrast to Tatsumi's material, which was synthesized in the presence of a chiral surfactant template, the C₁₈MIM-MSN was prepared by using an achiral surfactant (C₁₈MIMBr) as the structure-directing agent. As indicated by the arrow-pointed areas in FIG. 3c, each visible fringe represents the (100) interplanar spacing. The distance between two fringes is one-sixth of a pitch or a 60° rotation through the center of the long axis. It is noteworthy that all the particles shown in FIG. 3c appeared to have rotations of approximately 120° regardless the different particle sizes. The powder XRD analysis (FIG. 4c) of the C₁₈MIM-MSN material further confirmed the twisted hexagonal ordering of the mesopores as evidenced by the diffraction pattern of an intense (100) peak along with a well-resolved (110) and a broadened (200) peaks. The handedness of the rotation (right- or left-handed) could not be determined from the TEM analysis. As discussed in Tatsumi's report, the ratio of the left- and right-handedness of their chiral mesoporous silica material (65/35, left/right) was not entirely governed by the intrinsic chirality of the surfactant template since only the L-enantiomer of the chiral surfactant was employed. In the instant case, it was hypothesized that, as the alkyl chain lengths of the CₙMIMBr increases from C₁₄ to C₁₈, tighter intermolecular packing between the methylimidazolium head groups of the achiral CₙMIMBr molecules might have occurred. Given the planar structure of the imidazolium group, such tight packing would perhaps cause a staggered wadding of the CₙMIMBr molecules and twisted the micelles into a chiral structure.

This assumption was further investigated by the TEM and XRD analyses of the C₁₄OCMIM-MSN material that was synthesized with C₁₄OCMIMCl, which is structurally similar to the C₁₆MIMBr that gave rise to a MCM-41 type mesoporous structure. The mesoporous structure of the C₁₄OCMIM-MSN material appeared to be disordered as indicated by a broad XRD diffraction peak at 4.22° representing superimposed (110) and (200) peaks (FIG. 4d). The TEM micrograph shown in FIG. 3d is also consistent with this observation. Given that the hydrophilic polar region of C₁₄OCMIM, with the ether moiety close to the methylimidazolium head group, is significantly larger of that of C₁₆MIM, the results support our theory that the micellar structure and packing is strongly influenced by the alkyl chain length of the alkylimidazolium template.

Synthesis of RTIL compounds was performed as follows.

### Synthesis of 3-alkyl-1-methylimidazolium bromide (CₙMIMBr)

A 1-bromoalkane (50 mmol) was mixed with 1-methylimidazole (50 mmol, 4.1 g). The mixture was charged to a 100 mL flask, refluxed at 90° C. for 48 hours, and cooled to room temperature. The brown waxy substance obtained was recrystallized in THF twice. The pure white product was collected by filtration, and dried in vacuum at room temperature. The pure product was characterized by ¹H NMR.

### Synthesis of 1-chloromethoxytetradecane

To a 250 ml flask charged with paraformaldehyde (375 mg, 12.5 mmol) and 7.5 mL trimethylchlorosilane, 1-tetradecane (2.7 g, 12.5 mmol) was added slowly in small increments. The solution was stirred for two hours after it turned clear. The solvent was removed under reduced pressure and the product was characterized by ¹H NMR.

### Synthesis of 3-methoxytetradecyl-1-methylimidazolium chloride (C₁₄MIMCl)

A 100 ml flask was charged with 1-chloromethoxytetradecane (3.1 g, 11.8 mmol) and placed on ice. While on ice, 1-methylimidazole (1 g, 11.8 mmol) was added dropwise, and the mixture was refluxed for 18 hours, then was cooled to room temperature. The white waxy substance was dissolved in hot THF and crystallized. The crystallization procedure was repeated. The pure product was collected by filtration, and dried under vacuum at room temperature. The pure product was characterized by ¹H NMR.

Syntheses of antibacterial room-temperature ionic liquid templated mesoporous silica nanospheres (RTIL-MSN) were performed as follows.

The RTIL-MSNs were synthesized in a method similar to the following experimental description. One of skill in the art can make appropriate variations when necessary. A RTIL, such as 3-alkyl-I-methylimidazolium bromide (C₁₆MIMBr, 1.06 g, 2.74×10⁻³ mol) was first dissolved in 480 mL of Nanopure water. Aqueous sodium hydroxide (2.00 M, 3.5 mL) was added to the solution followed by adjusting the solution temperature to 353 K. Tetraethyl orthosilicate (5.00 mL, 2.24×10⁻² mol) was introduced quickly. This solution was allowed to stir for two hours at ambient temperature. This reaction gave rise to white precipitate. The precipitate was filtered, washed with deionized water and methanol, and lypholized. To remove the RTIL template, 400 mg of as-synthesized MSN was refluxed for 24 hours in a solution of 9 mL of HCl (12.1 M) and 200 mL of methanol.

Room temperature ionic liquids have been used as templates to synthesize unique mesoporous silica nanoparticles and the antibacterial activity of RTIL-MSNs has been measured against E. coli K12. The pore and particle morphologies are dependent on the RTIL used to template the MSN evidenced by small angle XRD, TEM, BET, and BJH analysis.

Instrument Methods, Conditions, and Parameters for the Structure Characterizations of Antibacterial RTIL Templated MSN

Powder XRD diffraction data were collected on a Scintag XRD 2000 X-ray diffractometer using Cu Kα radiation. The sample was scanned from 1.5° to 10° (2θ) with a step size of 0.02° and a count time of 0.5 s at each point. Nitrogen adsorption and desorption isotherm, surface area (SA), and median pore diameter were measured using a Micromeritics ASAP2000 sorptometer. Sample preparation included degassing at 363 K overnight. Nitrogen adsorption and desorption isotherms of these materials were obtained at 77 K. Specific surface areas and pore size distributions were calculated using the Brunauer-Emmett-Teller (BET) and Barrett-Joyner-Halenda (BJH) method, respectively. Particle morphology of these materials was determined by transmission electron microscopy (TEM) using a Phillips model CM30 TEM operated at 300 kV.

### Example 3

### Delivery of Antibacterial Agents

To study the mass-transport properties of these CₙMIM-MSN materials, we the controlled release profiles of these materials was investigated by utilizing the templating RTILs as antibacterial agents against the Gram (−) microbe Escherichia coli K12 as depicted in FIG. 2. Results indicated that the rates of release of the RTILs from the MSN materials are governed by the particle and pore morphology leading to different antibacterial activities.

It is widely known that cationic surfactants possess antibacterial properties, several can be found in household soaps and detergents (Davis, B.; Jordan, P. In Ind. Appl. Surfactants 2; Royal Society of Chemistry, 1990; Vol. 77, pp 195-210; Karsa, D. R., Ed.; Royal Society of Chemistry; Cambridge, 1990; Vol. 77, pp. 195-210). A recent report (Pemak, supra) has demonstrated the antibacterial activity of C₁₄OCMIMCl on both Gram (+) and Gram (−) microbes. The mechanism of the antibacterial activity of C₁₄OCMIMCl was attributed to the electrostatic interaction of phosphate groups on the microbial cell wall and the cationic methylimidazolium head group of the RTIL. Also, the organic tail region embeds itself in the lipid bilayer. This in turn leads to the free flow of electrolytes out of the microbe and causes the cell death. This is believed to be the mechanism of cell death for the other RTIL as well.

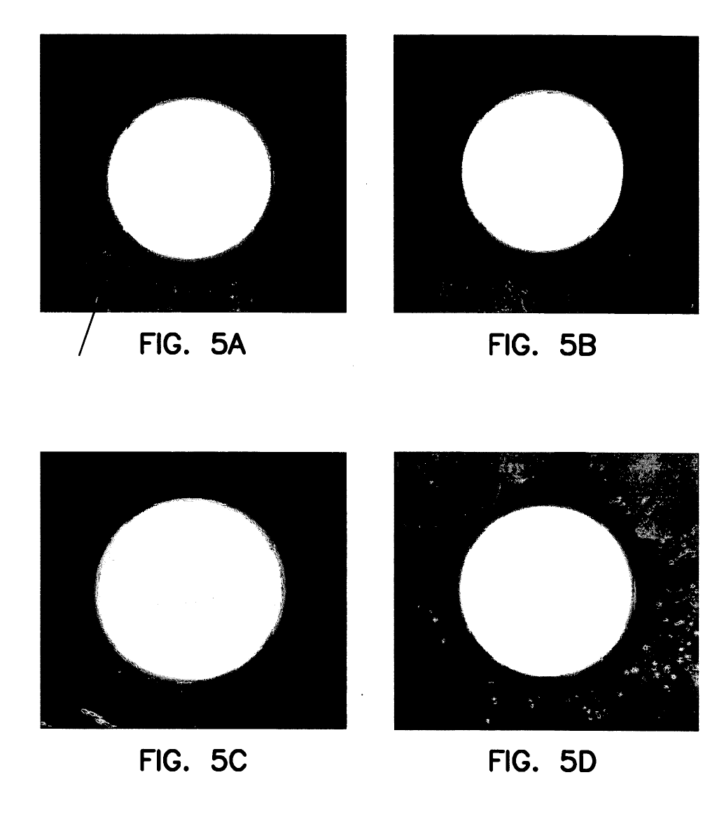

The antibacterial activity of the RTILs was measured by three methods: disk diffusion assays, minimal inhibitory concentration (MlC), and minimal bactericidal concentration (MBC). The disk diffusion assay was determined by placing a 25 mm cellulose disk saturated with 15 mM of C₁₆MIMBr, C₁₄OCMIMCl, and CPBr in phosphate buffer onto agar plates seeded with E. coli K12. As depicted in FIGS. 5a-d, the results of the disk diffusion assay showed an average of 35 mm of microbial clearing for C₁₆MIMBr, C₁₄OCMIMCl, and CPBr. The control (a cellulose disk saturated with 100 mM phosphate buffer pH 7.4) showed no antibacterial activity. The MIC and MBC concentrations were determined by dissolving ten different concentrations (10-100 □M) of C₁₆MIMBr, CPBr, and C₁₄OCMIMCl in broth media, inoculated in a 1:1 ratio with stock E. coli K12 culture, and visually determining the lowest concentration that lacked bacteria growth for the MIC. The MBC was measured by spreading one loopful from the tubes each dilution onto the agar plates and visually determining the lowest concentration of RTIL that supported no colony formation. The MIC of both RTILs was 30 μM. The MBC of the RTILs deviated slightly from one another. The MBC of C₁₆MIMBr was 100 μM and the MBC of CPBr and C₁₄OCMIMCl was 70 μM.

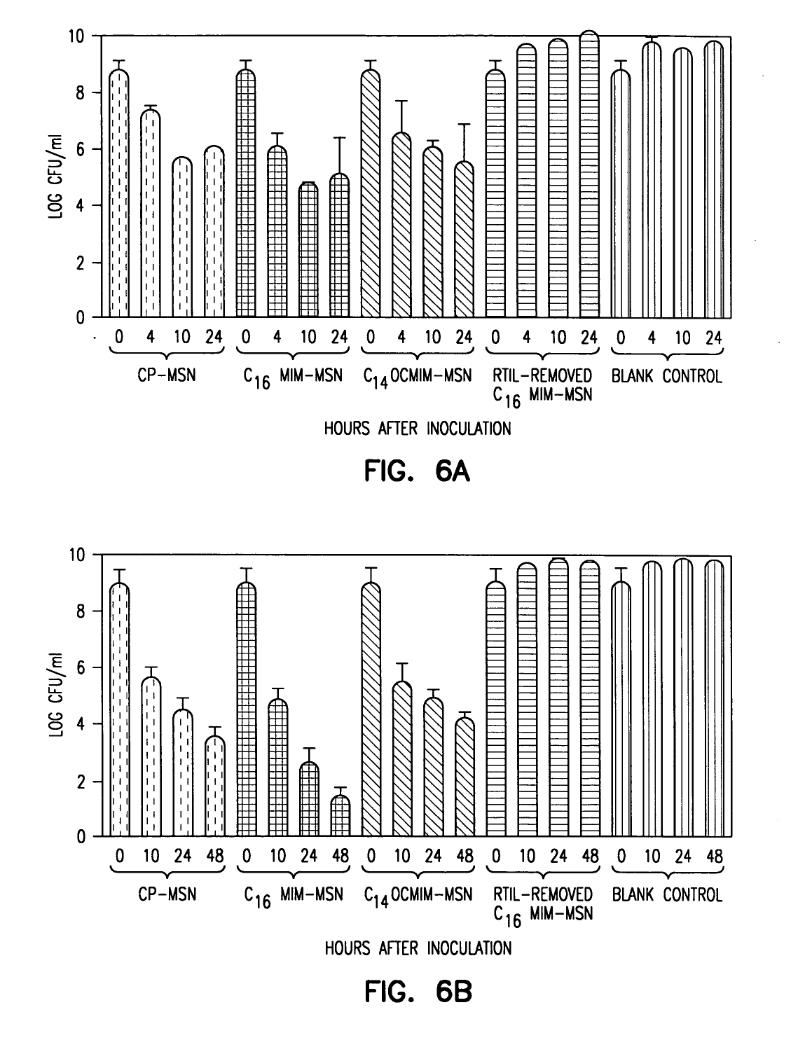

The antibacterial activities of CP-MSN, C₁₆MIM-MSN, and C₁₄OCMIM-MSN (the preparation of which was described in Example 2), were measured by series dilution for 24 hours at two temperatures (25° C. and 37° C.) as seen in FIGS. 6a and b, respectively. The two MSNs were suspended in 5 ml of tryptic soy broth with 0.6% yeast extract and inoculated with 1.0 mL of 18 hour stock culture of E. coli K12. At various times aliquots of each sample were diluted and plated on tryptic soy agar with 0.6% yeast extract. The plates were incubated for 18 hours. Colonies were counted and recorded for dilutions containing between 30 and 300 colonies. Contradictory to the MBC results of free RTILs, C₁₆MIM-MSN exhibited a better antibacterial activity than that of C₁₄OCMIM-MSN by a thousand fold. The diffusion of both RTIL from the pores slowed down at 25° C. It is reasonable that the microbial killing activity of the two RTIL-MSNs deviated more when diffused from the pores rather than in solution. According to the TEM measurements the pore morphologies of these two samples are very different. C₁₆RMIM-MSN has a hexagonal array ordered pores that all line up parallel with a spherical morphology, while C₁₄OCMIM-MSN has a disordered pore arrangement with a curved cylindrical shape. In addition to pore morphology, the mass transfer of RTIL from the tubular particles (C₁₄OCMIM-MSN) will be considerably slower than the spherical particle (C₁₆MIM-MSN).

The antibacterial activity was dependent on the rate of diffusion of the RTIL, which was dependent on the particle and pore morphology. Further work is continuing to measure the effect of interior and exterior functionalization on antibacterial activity of RTIL-MSN.

Bacterial Culture.

Microbial media used in these experiments included trypticase soy broth with 0.6% yeast extract and tryptic soy agar with 0.6% yeast extract. The microorganism used was Escherichia coli K12 purchased from Fluka. Broth cultures were grown at 37° C. in a shaker incubator for 18 hours and plated cultures were grown at 37° C. in a static incubator for 18 hours unless otherwise reported.

Disk Diffusion Assay

Tryptic soy agar plates were seeded with 200 μL, 18 hour stock E. coli K12 cultures. Stock solutions of 15 mM C₁₆MIM-MSN and C₁₄OCMIM were prepared in 100 mM phosphate buffer, pH 7.4. These solutions were used to saturate 25 mm cellulose disks. These disks, along with a negative control (buffer lacking RTIL), were placed in the center of the previously seeded plates, and incubated for 18-24 hours at 37° C. The diameters of the zones of complete inhibition were measured to the nearest whole millimeter.

Antimicrobial Activity of RTIL

Antimicrobial activity of the RTIL was determined by the tube dilution method. A series of C₁₆MIM-MSN and C₁₄OCMIM-MSN dilutions were prepared in trypticase soy broth with 0.6% yeast extract. A suspension of E. coli K12, prepared from a 24 hour culture, was added to each dilution in a 1:1 ratio. Growth (or the lack there of) of the E. coli was determined visually after incubation for 24 hours at 37° C. The lowest concentration at which there was no visible growth was taken as the MIC. From each tube one loopful was cultured on TSA with 0.6% yeast extract plates and incubated for 48 hours at 37° C. The lowest concentration of RTIL supporting no colony growth was defined as the MBC.

Antimicrobial Activity of RTIL-MSNs

Antimicrobial activity was determined by the tube dilution method at 37° C. and 25° C. A series of RTIL-templated MSNs (2.0 g) were prepared in broth. These five mL suspensions were inoculated with 1.0 mL stock, 18 hour culture. The four cultures prepared were C₁₆MIM-MSN, C₁₄OCMIM-MSN, acid washed C₁₆MIM-MSN, and a blank containing no silica material. These cultures were in turn incubated for zero, four, ten, twenty, and twenty-four hours. After the required time a dilution series was carried out to determine the growth in each culture. Plates were grown for 18 hours and colonies were counted and recorded for dilutions containing between 30 and 300 CFU. These set of experiments were repeated four times at 37° C. and three times at 25° C. The colony forming units at each measurement for each sample were averaged and a standard deviation was used to determine error. Outliers were determined and removed using Chauvenet's Criterion.

### Example 4

### Prevention/Elimination of Oral Malodor

A change in the balance of Gram (+) and Gram (−) bacteria can cause significant oral malodor. The oral cavity is a dynamic environment in a constant state of equilibrium, with both gram (+) and gram (−) bacteria existing in a healthy mouth. When this balance is shifted to predominantly gram (−) bacteria, the process for the formation of volatile sulfur compounds (VSCs) can begin. Gram (+) bacteria break down carbohydrates in an aerobic fashion. Gram (−) bacteria, on the other hand, operate in an anaerobic fashion. When gram (+) bacteria run out of fuel, typically in the form of carbohydrates, the balance can shift to gram (−) bacteria.

Once the balance is shifted to gram (−) bacteria, the pH of the mouth rises from below ˜6.5 to >7.2. Maintaining a pH below 6.5 will inhibit the gram (−) bacteria from breaking down protein. Since gram (−) bacteria operates in an anaerobic fashion, areas of stagnant saliva are needed (dry mouth). This is one reason that bad breath is more prevalent in the morning than at other times of the day. The presence of protein, in the form of exfoliated epithelium, leukocytes, food debris, and dead bacteria, or a combination thereof, serves as fuel for the formation of VSCs. Once the proper conditions are established, stagnant pools of saliva containing protein, anaerobic breakdown can begin.

Protein is broken down by proteolysis to form peptides and further into amino acids and then to VSCs. The amino acids found most responsible for the formation of VSCs were cysteine and methionine. Each of these amino acids contain sulfur groups that when broken down form H₂S and CH₃SH. It was found that the main contributors to oral malodor are these by-products. The formation of VSCs will continue until the environmental conditions are changed and the balance of gram (+) and gram (−) bacteria is restored.

Gas chromatography/mass spectrometry (GC/MS) can be used as a screening tool for measuring the amount of VSCs generated in a person's mouth. By testing a sample of their breath, the quantity of VSCs can be determined. This analytical tool can be used to evaluate methods to determine which MSN particle composition has the greatest impact on reducing oral malodor.

It is known that zinc ions reduce the VSC production in oral cavity. The mechanism involved a reaction between sulfur-containing substrates and zinc yielding non-volatile metal sulfide compounds. This process inhibits the generation of VSCs. Mouth rinses containing zinc salts with cationic bis-biguanidines and quaternary ammonium antibacterial agents, such as chlorhexidine and cetylpyridinium chloride, respectively, have been widely used in preventive dentistry as effective inhibitors of plague formation and of development of gingivitis. However, in order to obtain consistent clinical effectiveness, it is necessary to apply certain concentrations (>0.2%) of aqueous solution of these bitter-tasting compounds at least twice daily. Such requirements of concentration and frequency of application are often overlooked or ignored by patients. It would be beneficial to design a non-toxic delivery material/system that can adhere to gum line and release these VSC-inhibitory compounds in a controlled fashion.

Described herein is a series of recently developed Mesoporous Silica Nanosphere (MSN) materials as a controlled release carrier system that can encapsulate and interactively release the aforementioned VSC-inhibitory chemicals when the oral pH changes to a VSC-prone condition.

(1) Synthesis of Antibacterial Agent-Containing MSN Material with Zinc Binding and Releasing Capability:

A series of novel amino acid-functionalized, cetylpyridinium chloride-containing MSN materials has been prepared and characterized. These monodisperse materials are either spherical or rod-shaped with an average particle size of 500 nm. As depicted in FIG. 2, the nanometer-sized pores are filled with the aforementioned antibacterial agent, cetylpyridinium chloride/bromide (CPC) molecules. The pore surface can also be functionalized with a series of zinc-binding amino acids, such as glutamic acid (Glu), histidine (His), and aspartic acid (Asp) groups. In addition, other CPC-binding amino acid groups, such as tryptophan, can also be covalently incorporated. The pores of the MSN can be functionalized with 3-[2-(2-aminoethylamino)ethylamino]propyl (AEP) groups, producing an AEP-functionalized, cetylpyridinium-containing MSN particle. Other groups that can be co-condensed in the MSN using a trialkoxy-silane include 3-aminopropyl (AP), N-(2-aminoethyl)-3-aminopropyl (AAP), ureidopropyl (UDP), 3-(ICP), 3-cyanopropyl (CP), and allyl (AL). Metals, metal ions, or metal compounds (for example, zinc acetate) can be loaded into the MSN particles by the method described in Example 5. Both cetylpyridinium and zinc ions can be released at acidic pH condition.

In neutral and weakly basic conditions (pH 7.0 to 8.5), the CPC molecules will slowly diffuse out of the pores of our MSN materials. The typical strong electrostatic attraction between the CPC and silica surface is significantly hindered due to the presence of these amino acid-zinc complexes on the mesoporous silica surface. CPC-releasing materials can suppress the anaerobic protein digestion activities of the gram (−) microorganisms in saliva, and thereby eliminate the VSC formation. Furthermore, the pore surface-anchored amino acids can also bind to zinc ions in neutral pH aqueous solutions either through metal-ligation or electrostatic attraction. The ligand-metal bonding or electrostatic force between the aforementioned surface-bound amino acids and zinc are not very strong. The zinc-ligation abilities of the major VSC-prone chemicals, such as methionine and cysteine, are orders of magnitude stronger than the MSN bound His, Glu, and Asp groups. Therefore, the methionine and cysteine generated by gram (−) bacteria will be able to competitively bind to the MSN surface adsorbed zinc ions. By concomitantly releasing antibacterial agents and inducing the binding of VSC-prone chemicals to zinc ions on MSN surface, MSN system can effectively eradicate the VSC-related oral malodor problem.

(2) Fine-Tuning the Amount and Kinetics of Release of CPC and Zinc Ions:

To determine the ideal chemical composition of MSN materials that can optimize the release efficiency of CPC/zinc, one can systematically screen the aforementioned amino acids for the best performing functionality that can encapsulate and release an optimal amount of VSC-inhibitory agents (CPC) with the desired rate of release. The loading and chemical nature of zinc-binding amino acids per unit weight of MSN solid can also be fine-tuned, so that the removal of methionine and cysteine via the zinc binding interaction can be adjusted. In vitro experiments using human saliva can be conducted to simulate the in vivo conditions. Depending on the results of these experiments, the pore surface of the MSN materials can be further derivatized to gain additional control of CPC release.

(3) Coating of Exterior Surface of MSN Materials with Dental Adhesives:

To prolong the lifetime of malodor-eliminating MSN when applied in mouth, the exterior surface of MSN can be coated with any of several widely used adhesives, such as, e.g., alkyl vinyl ether-maleic copolymers or poly(N-isopropylacrylamide). The exterior coating can allow a strong and long-lasting attachment of the MSN nanoparticles to epithelial cells at the gum line and thereby enhance the effectiveness of the system.

### Example 5

### Agent Loading Procedure

To remove the surfactant templates from the pores of MSNs, as-synthesized MSN can be refluxed for 24 hours in a solution of HCl and methanol (about 1.5M or about 1.8M solution). After removal of the surfactant template, antimicrobial agents can be added to the internal MSN pores by any suitable and effective means. One suitable method is to add purified MSNs to an ethanol solution containing the antimicrobial agent, followed by stirring the solution for 20 hours, during which time the MSNs adsorb the antimicrobial agents into the pores. The resulting MSNs with antimicrobial agents adsorbed into the pores are then filtered and washed with ethanol, methanol, and acetone, followed by drying under high vacuum.

The MSN-antimicrobial agent particles can then be further modified by post-synthesis grafting of a polymer to the surface of the MSNs, as described below in Example 6. Polymer modification thus converts the MSN-antimicrobial particles into delayed-release drug-delivery particles.

### Example 6

### Forming a Polymer Coating

Polymers can be covalently bonded to the surface of the mesoporous silicate particles of the invention. Such polymers can act as adhesives to adhere the particles to targeted areas on the body of a patient, or they can act as a diffusion barrier that prolongs the release of antimicrobial agents from the pores of the particles. Methods that can be used for attaching polymers to the surface of the MSNs have been described by, for example, Radu, et al., J. Am. Chem. Soc., 2004, 126 (6), 1640 -1641.

Described below is the synthesis and characterization of a poly(lactic acid)-coated, MCM-41-type mesoporous silica nanosphere (PLA-MSN) material that can serve as a delayed-release antimicrobial agent delivery system under physiological conditions. The PLA layer can be used as a gatekeeper to regulate the exit of molecules in and out of the nanoscale pores.

A mercaptopropyl-functionalized mesoporous silica nanosphere (thiol-MSN) material with average pore diameter of 2.5 nm was prepared via our previously reported method (Lin, V. S.-Y, et al., J. Am. Chem. Soc. 2001, 123, 11510-11511; Lai, C.-Y, et al, J. Am. Chem. Soc. 2003, 125, 4451-4459). The polymer linking moiety, 5,6-epoxyhexyltriethoxysilane (EHTES) was grafted onto the exterior surface of the thiol-MSNs containing cetyltrimethylammonium bromide (CTAB) surfactants inside the mesopores. The resulting material (1.50 g) was refluxed in a 162 mL methanol solution of hydrochloric acid (1.57 M) for 12 hours to remove the CTAB template and to convert the thiol-MSN with epoxyhexyl groups to a 5,6-dihydroxyhexyl-coated thiol-MSN material (DH-MSN). Incorporation of the 5,6-dihydroxyhexyl group was confirmed by ²⁹Si and ¹³C CP- and DP-MAS NMR spectroscopy, and the surface coverage was measured to be 43% (2.1 mmol/g). The vacuum-dried DH-MSN material (0.68 g) was sonicated for 30 minutes in 10 mL of anhydrous THF to disperse the particles uniformly. L-Lactide (0.36 g, 2.50 mmol) was mixed with a catalyst, tin(II) 2-ethylhexanoate (Sn(Oct)₂, 0.16 mL, 0.50 mmol), in 15 mL of anhydrous THF. The lactide/catalyst solution was added to the DH-MSN THF suspension via injection and stirred at 80° C. for 72 hours to yield the PLA-coated thiol-MSN material. The crude solid product was further purified by a method previously published by Langer's group (Choi, I. S.; Langer, R. Macromolecules 2001, 34, 5361-5363). The average thickness (ca. 11 nm) of the PLA layer was determined by transmission electron microscopy (TEM). The layer of PLA could be identified by the rim of amorphorous structure surrounding the MCM-41-type MSN core with mesopores packed in a hexagonal symmetry. The chemically accessible thiol density (0.22 mmol/g) of the purified PLA-MSN was measured by our previously published method (Lin, V. S.-Y, et al., J. Am. Chem. Soc. 2001, 123, 11510-11511). The mercaptopropyl functionality was then converted to an amine-sensitive OPTA group by reacting 85.0 mg of PLA-coated thiol-MSN with 170.0 mg (1.26 mmol) of phthalic dicarboxaldehyde (o-phthalaldehyde, OPA) in 10 mL of methanol solution for 5 hours. After filtration, the resulting material (PLA-MSN) was thoroughly washed with methanol and dried under vacuum. The morphology, particle size distribution, and the structure of organic functionalities of PLA-MSN were scrutinized by XRD, SEM, TEM, N₂ sorption isotherms, and ¹³C CP-MAS NMR spectroscopy.

To examine the gatekeeping effect of the PLA layer in our PLA-MSN system, we prepared and characterized an amorphous silica material grafted with the same OPTA functionality (OPTA-SS) as a control system. The surface coverage of the OPTA group was determined to be 0.08 mmol/g. Both the OPTA-SS and PLA-MSN materials were dispersed in pH 7.4 PBS buffer (10 mM) for the fluorescence-sensing experiments of neurotransmitters. In the case of OPTA-SS, dopamine, tyrosine, and glutamic acid (230 μM each) reacted with the surface-bound OPTA groups rapidly, as evidence by fluorescence emission data. It is noteworthy that both tyrosine and glutamic acid reacted to the OPTA-SS with very similar rates and therefore could not be distinguished from each other. In contrast, the reactions of these analytes (230 μM) with our OPTA-derivatized PLA-MSN exhibited significantly different and lower reaction rates, by a factor of 4, 10, and 57, respectively. In the case of dopamine, the lower reaction rate could be attributed to the additional diffusional penetration through the PLA layer into the OPTA-functionalized mesopores. Clearly, the reaction rates of tyrosine and glutamic acid were further slowed by the gatekeeping effect of the PLA layer on these two analytes. In addition, analysis showed that the fluorescence intensity of OPTA-SS increased similarly with the increasing concentrations of all three neurotransmitters. However, in the case of the PLA-MSN, the dopamine binding gave the most significant increase of fluorescence intensities at all concentrations. A similar set of kinetic and titration experiments performed on the DH-MSN material (without PLA) showed no evidence of the gatekeeping effect.

To examine the substrate selectivity of the PLA-MSN system in the presence of a mixture of neurotransmitters, PLA-MSN nanoparticles (2 mg) were introduced to a pH 7.4 PBS buffer (10 mM) solution of dopamine (0.5 mM) and glutamic acid (10 mM) at 25° C. After 10 minutes of mixing, the suspension was centrifuged, and the individual concentrations of dopamine and glutamic acid in the supernatant were analyzed by HPLC. Given that the signal transduction mechanism of the PLA-MSN system is based on the covalent capture of substrates by the surface-bound OPTA groups, the different degrees of concentration decrease of these two analytes in solution would represent the selectivity of the PLA-MSN system. Despite the initial 20:1 concentration ratio between glutamic acid and dopamine, the results showed a 96% decrease of dopamine concentration, whereas only a 2% decrease of the concentration of glutamic acid was observed.

The observed large difference in the rates of diffusion is most likely due to the different electrostatic, hydrogen bonding, and dipolar interactions between these neurotransmitters and the PLA layer in pH 7.4 buffer. The isoelectric points (pIs) of dopamine, tyrosine, and glutamic acid are 9.7, 5.7, and 3.2, respectively, whereas the pI of PLA is typically below 2.0, which means the dopamine will be positively charged and the others will be negatively charged under our experimental conditions. Similar effects of pI have also been reported. For example, Blanco et. al (Eur. J. Pharm. Biopharm. 1998, 45, 285-294) reported that proteins with low pI values, such as bovine serum albumin (pI=4.6) were released faster from a PLGA-based polymer than those with high pls, such as lysozyme (pI=11.2).

The gatekeeping effect of the PLA-MSN system can also be used to prepare prolonged-release antimicrobial agent delivery systems by loading antimicrobial agents into the pores of the MSNs before forming the PLA coating. The PLA coating can serve to regulate the diffusion of antimicrobial agents from the pores of the PLA-MSN to the targeted area of a patient. Other organic functionality can be grafted to the surface of the MSNs by the methods described by, for example, Lin, V. S.-Y. et al., J. Amer. Chem. Soc. 2001, 123, 11510-11511.

### Example 7

### Dosage Forms

The following illustrate representative pharmaceutical dosage forms, containing a loaded mesoporous silica particle of the invention (‘Particle X’), for therapeutic or prophylactic use in mammals.

| (i) Tablet 1 | mg/tablet |
|---|---|
| ‘Particle X’ | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |
| (ii) Tablet 2 | mg/tablet |
| ‘Particle X’ | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| ‘Particle X’ | 10.0 |
| Colloidal silicon dioxide | 1.5 |
| Lactose | 465.5 |
| Pregelatinized starch | 120. |
| Magnesium stearate | 3.0 |
| | 600.0 |

| (iv) Injection 1 (1 mg/mL) | mg/mL |
|---|---|
| ‘Particle X’ (free acid form) | 1.0 |
| Dibasic sodium phosphate | 12.0 |
| Monobasic sodium phosphate | 0.7 |
| Sodium chloride | 4.5 |
| 1.0 N Sodium hydroxide solution | q.s. |
| (pH adjustment to 7.0-7.5) | |
| Water for injection | q.s. ad 1 mL |
| (v) Injection 2 (10 mg/mL) | mg/mL |
| ‘Particle X’ (free acid form) | 10.0 |
| Monobasic sodium phosphate | 0.3 |
| Dibasic sodium phosphate | 1.1 |
| Polyethylene glycol 400 | 200.0 |
| 0.1 N Sodium hydroxide solution | q.s. |
| (pH adjustment to 7.0-7.5) | |
| Water for injection | q.s. ad 1 mL |

| (vi) Aerosol | mg/can |
|---|---|
| ‘Particle X’ | 20.0 |
| Oleic acid | 10.0 |
| Trichloromonofluoromethane | 5,000.0 |
| Dichlorodifluoromethane | 10,000.0 |
| Dichlorotetrafluoroethane | 5,000.0 |

The above formulations may be obtained by conventional procedures well known in the pharmaceutical art.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A mesoporous silicate body having one or more pores;
one or more room temperature ionic liquid (RTIL) cations within one or more of the pores of the mesoporous silicate body; and
one or more functionalized organic groups covalently bonded to the one or more pores.

2. The body of claim 1 wherein the body is a RTIL-templated mesoporous silicate body.

3. The body of claim 1 wherein the RTIL cation is an antimicrobial agent.

4. The body of claim 1 further comprising an antimicrobial agent.

5. The body of claim 1 wherein the functionalized organic group comprises an alkyl thiol.

6. The body of claim 1 wherein the functionalized organic group comprises one or more amino acids.

7. The body of claim 6 wherein the one or more amino acids are selected from the group consisting of glutamic acid, histidine, and aspartic acid.

8. The body of claim 3 wherein the antimicrobial agent is a higher(alkyl)pyridinium cation.

9. The body of claim 8 wherein the antimicrobial agent is a cetylpyridinium cation.

10. The body of claim 3 wherein the antimicrobial agent is a 1-higher(alkyl)-3-alkylimidazolium cation.

11. The body of claim 10 wherein the antimicrobial agent is a cation selected from the group consisting of 1-tetradecyl-3-methylimidazolium, 1-hexadecyl-3-methylimidazolium, 1-octadecyl-3-methylimidazolium, 1-tetradecyloxymethyl-3-methylimidazolium, and a combination thereof.

12. The body of claim 1 wherein the one or more pores of the mesoporous silicate body have an average pore diameter of about 1 to about 4 nm.

13. The body of claim 4 wherein the antimicrobial agent is effective against cocci, rods, or fungi.

14. The body of claim 4 wherein the antimicrobial agent is effective against gram negative bacteria, gram positive bacteria, or both.

15. The body of claim 1 that has a spheroid shape, ellipsoid shape, a rod-like shape, or a curved cylindrical shape.

16. The body of claim 1 wherein the RTIL cations diffuse from the pores when it is in contact with a liquid that has a pH of greater than about 7.

17. The body of claim 1 wherein the RTIL cations diffuse from the pores when it is in contact with a liquid that has a pH of about 7.5 to about 9.

18. The body of claim 1 further comprising a polymer covalently bonded to the surface of the mesoporous silicate body.

19. The body of claim 3 further comprising a polymer covalently bonded to the surface of the mesoporous silicate body.

20. The body of claim 4 further comprising a polymer covalently bonded to the surface of the mesoporous silicate body.

21. The body of claim 18 wherein the polymer slows the rate of diffusion of the RTIL cations from the pores of the mesoporous silicate body when it is in contact with a liquid.

22. The body of claim 18 wherein the polymer is an adhesive.

23. The body of claim 22 wherein the adhesive adheres the body to the oral tissue of a mammal when the body is contacted with the oral tissue of said mammal.

24. The body of claim 22 wherein the adhesive adheres the body to the skin or to a mucus membrane of a mammal when the mesoporous body is contacted with the cells or the membrane.

25. The body of claim 22 wherein the adhesive is poly(N-isopropylacrylamide), an alkyl vinyl ether-maleic copolymer, or both.

26. The body of claim 1 that can bind and release metal ions or metal-containing compounds.

27. The body of claim 1 wherein the body comprises one or more metals, metal compounds, metal cations, bis-biguanidines or salts thereof.

28. The body of claim 27 wherein the metal cations comprise zinc cations.

29. The body of claim 27 wherein the metal compound comprises zinc acetate.

30. The body of claim 27 wherein the bis-biguanidines comprise chlorhexidine, or salts thereof.

31. A pharmaceutical composition comprising an effective amount of the bodies of any one of claims 1, 3, 4, and 18, in combination with a pharmaceutically acceptable diluent or carrier.

32. A cosmetic composition comprising an effective amount of the bodies of any one of claims 1, 3, 4, and 18, in combination with a cosmetically acceptable diluent or carrier.

33. A method of treatment comprising inhibiting microbial growth by contacting a mammal with an effective amount of a population of bodies of claim 3 or 4.

34. The method of claim 33 wherein the mammal is a human.

35. The method of claim 33 wherein the bodies are contacted with the skin or a mucus membrane of the mammal.

36. The method of claim 33 wherein the bodies are contacted with the oral tissue of the mammal.

37. The method of claim 36 wherein the treatment reduces the amount of volatile sulfur compounds in the mouth.

38. A method for synthesizing mesoporous silicate nanoparticles comprising
co-condensing one or more tetraalkoxy-silanes and one or more room temperature ionic liquids (RTILs) so as to provide a population of mesoporous silicate particles having monodisperse particle sizes,
wherein the RTIL is not a co-solvent, and
wherein the nanoparticles are ellipsoid-, rod-, or tubular-shaped.

39. The method of claim 38 wherein the mesoporous silicate particles are prepared by co-condensing one or more tetraalkoxy-silanes and a 1-hexadecyl-3-methylimidazolium salt to provide the mesoporous silicate particles as ellipsoids.

40. The method of claim 38 wherein the mesoporous silicate particles are prepared by co-condensing one or more tetraalkoxy-silanes and a 1-octadecyl-3-methylimidazolium salt to provide the mesoporous silicate particles as rods.

41. The method of claim 38 wherein the mesoporous silicate particles are prepared by co-condensing one or more tetraalkoxy-silanes and a 1-tetradecyloxymethyl-3-methylimidazolium salt to provide the mesoporous silicate particles as curved cylindrical shaped particles.

42. The method of claim 38 further comprising co-condensing one or more organo-substituted trialkoxy-silanes.

43. The method of claim 42 wherein the organo-substituted trialkoxy-silane is an thioalkyl-substituted trialkoxy-silane.

44. A method of delivering an antimicrobial agent to a mammal comprising:
contacting the mammal with an effective amount of RTIL-templated mesoporous silicate particles that contain an antimicrobial quaternary ammonium cation within one or more pores.

45. The method of claim 44 wherein the mammal is a human.

46. The method of claim 44 wherein the antimicrobial agent is a higher(alkyl)pyridinium cation.

47. The method of claim 44 wherein the antimicrobial agent is cetylpyridinium.

48. The method of claim 44 wherein the antimicrobial agent is a 1-higher(alkyl)-3-alkylimidazolium cation.

49. The method of claim 44 wherein the antimicrobial agent is a cation selected from the group consisting of 1-tetradecyl-3-methylimidazolium, 1-hexadecyl-3-methylimidazolium, 1-octadecyl-3-methylimidazolium, 1-tetradecyloxymethyl-3-methylimidazolium, and combinations thereof.

50. The method of claim 44 wherein the mesoporous silicate particles can bind and release metal ions or metal-containing compounds.

51. The method of claim 44 wherein the mesoporous silicate particles comprise zinc-binding amino acids selected from the group consisting of glutamic acid, histidine, aspartic acid, and a combination thereof.

52. The method of claim 44 wherein the mesoporous silicate particles comprise one or more metals, metal compounds, metal cations, bis-biguanidines, or salts thereof.

53. The method of claim 52 wherein the metal cations comprise zinc cations.

54. The method of claim 52 wherein the metal compound comprises zinc acetate.

55. The method of claim 52 wherein the bis-biguanidine is chlorhexidine, or a salt thereof.

56. The method of claim 44 wherein skin or a mucus membrane of the mammal is contacted with the mesoporous silicate particles.

57. The method of claim 44 wherein the oral tissue of the mammal is contacted with the mesoporous silicate particles.

58. The method of claim 57 wherein the treatment reduces production of volatile sulfur compounds in the mouth of the mammal.

59. The method of claim 44 wherein the antimicrobial agent is effective against cocci, rods, or fungi.

60. The method of claim 44 wherein the antimicrobial agent is effective against gram negative bacteria, gram positive bacteria, or both.

61. The method of claim 44 wherein the antimicrobial agent is selective for a specific bacteria or fungus.

62. The method of claim 44 further comprising a polymer covalently bonded to the surface of the mesoporous silicate bodies.

63. The method of claim 62 wherein the polymer slows the rate of diffusion of the antimicrobial agent from the pores of the mesoporous silicate bodies when they are in contact with a liquid.

64. The method of claim 62 wherein the polymer is an adhesive that adheres the bodies to oral tissue of a mammal when the bodies are contacted with the oral tissue of a mammal.

65. The method of claim 62 wherein the polymer is an adhesive that adheres the bodies to skin or to a mucus membrane of a mammal when the bodies are contacted with skin or a membrane.

66. The method of claim 64 or 65 wherein the adhesive is poly(N-isopropylacrylamide), an alkyl vinyl ether-maleic copolymer, or both.

67. An antimicrobial delivery system that allows for delayed release of antibacterial agents from a single application of mesoporous silicate particles, comprising:
a population of mesoporous silicate particles having one or more pores, and
one or more antimicrobial agents within one or more pores,
wherein the mesoporous silicate particles release the antimicrobial agents from the pores over an extended period of time.

68. The antimicrobial delivery system of claim 67 further comprising one or more amino acids covalently bonded to the pores or the surface of the mesoporous silicate particles, wherein the amino acid influences the release rate of an antimicrobial agent.

69. The antimicrobial delivery system of claim 67 wherein the antimicrobial agent is selective for gram negative bacteria, gram positive bacteria, or both.

70. The delivery system of claim 67 further comprising a polymer that is covalently bonded to the surface of the mesoporous silicate particles.

71. The delivery system of claim 70 wherein the polymer is poly(N-isopropylacrylamide), an alkyl vinyl ether-maleic copolymer, or both.

72. The delivery system of claim 70 wherein the polymer is poly(lactic acid).

73. A method of reducing oral volatile sulfur compounds comprising contacting a mammal with an antimicrobial controlled-release composition which comprises one or more bodies of claim 18, 19, or 20.
